(19) United States
(12) Patent Application Publication
(10) Pub. No.: US 2023/0240953 A1
(43) Pub. Date: Aug. 3, 2023

(54) AN ANTIMICROBIAL COMPOSITION, PROCESS FOR PREPARING THE SAME AND METHOD OF USE THEREOF

(71) Applicant: ISP INVESTMENTS LLC, Wilmington, DE (US)

(72) Inventors: Solomon Howard JACOBSON, Berkeley Heights, NJ (US); Yevgenly TUROVSKIY, Somerville, NJ (US); Joseph TORELLA, Metuchen, NJ (US); Karen WINKOWSKI, Springfield, NJ (US)

(73) Assignee: ISP INVESTMENTS LLC, Wilmington, DE (US)

(21) Appl. No.: 18/011,903

(22) PCT Filed: Jun. 28, 2021
(86) PCT No.: PCT/US21/39307

§ 371 Date: Dec. 21, 2022

Related U.S. Application Data
(60) Provisional application No. 63/046,417, filed on Jun. 30, 2020.
(60) Provisional application No. 63/089,382, filed on Oct. 8, 2020.

(51) Int. Cl. A61K 8/35

(52) U.S. Cl. CPC A61K 8/35; A61K 2800/74; A61K 2800/524

(57) ABSTRACT

The present application provides an antimicrobial composition comprising: (a) about 0.01 to about 99.9 wt. % of at least one compound having a structure of formula (I) or formula (II):

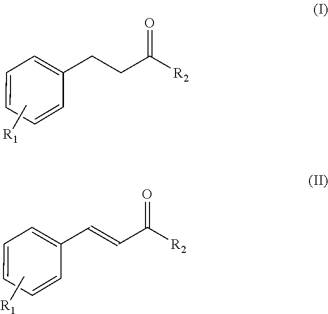

wherein R₁ is hydrogen, alkyl, alkoxy or hydroxyl; R₂ is hydrogen, alkyl or alkoxy; and (b) about 0.1 to about 95% wt. % of one or more antimicrobial compounds not being compounds of formula (I) and formula (II). Also disclosed is a process for preparing the compositions and method of use thereof.

### FIELD OF THE INVENTION

The present application relates to an antimicrobial composition, and, more particularly, to an antimicrobial composition comprising: (a) 0.01 to 99.9 wt. % of at least one compound having a structure of formula (I) or formula (II) defined below; and (b) about 0.1 to about 95% wt. % of one or more antimicrobial compound not being compounds of formula (I) and formula (II). Also disclosed is a process for preparing the compositions and method of use thereof.

### BACKGROUND OF THE INVENTION

Commercial use products are generally designed to have a substantial shelf life. The products need to be manufactured at one site, transported possibly over a considerable distance to a depot or other storage facility prior to further transport to a point of sale. The product may then spend considerable time on a retailer's shelf prior to purchase and further storage by the user whether for individual use or use in, for example, a workplace, institution or the like. Storage typically takes place under uncontrolled conditions including considerable variation in temperature. In order to keep bacterial and fungal growth in such products at an acceptable level, it is conventional practice for the products to contain a preservative. Many preservatives are available. The appropriate preservative needs to be selected based on its efficacy and its acceptability in the product.

Classic preservatives such as formaldehyde donors, parabens, and isothiazolinones are generally very effective at controlling microorganisms in various personal care products. However, in recent years, many consumers developed unfavorable perception of these chemistries. Current market trends strongly favor safe, non-irritating, natural/nature-identical, and biodegradable actives. As a result of these changes, there is a strong need for development of novel antimicrobial technologies for personal and home care applications. Chemistries that can boost antimicrobial performance of known actives are highly desired.

There are multiple benefits in using a combination of antimicrobials, especially if they act synergistically with one another. Firstly, the use of such combination greatly reduces the risk of microorganisms developing resistance to the antimicrobials. Additionally, synergistically acting antimicrobials can be used in lower concentrations thus reducing the cost. Finally, a synergistically acting multi-component antimicrobial system is likely to be active versus a broader range of microorganisms, or to provide a faster kill rate, compared to each component acting individually. For these reasons, identifying synergistic combinations of antimicrobial actives is critical for development of effective preservative systems.

KR Patent 1881306B1 discloses cosmetics, personal care products, and home care and fabric care compositions comprising raspberry ketone, an alkyl-arginine derivative and 1,2-decanediol.

US Publication 20080317681 discloses a composition used as a chewing gum or confectionary for removing stains and microbes from teeth of warm-blooded animals having a stain removing complex containing stain removing agent and a cyclodextrin compound, and optionally a gum base.

US Publication 20170096381 discloses the use of vanillin derivatives in cosmetic, dermatological or pharmaceutical compositions.

PCT Application 2010084661A1 discloses an isothiocyanate containing composition for treating insulin-like growth factor-1 associated diseases, e.g., hair loss and dementia, comprising isothiocyanates and one or more of isoflavone, raspberry ketone, capsiate and gluconic acid.

KR publication 2006034941A discloses an acaricidal composition is to control mites by selective insect killing and easy biodegradation of the compounds comprising at least one compound having acaricidal activity selected from 2-methoxyphenylacetone, 4-methoxyphenylacetone and 4-(4-methoxyphenyl)-2-butan-2-one.

In view of the foregoing, still there is a need for improved antimicrobial compositions for aqueous or non-aqueous based end-user compositions for reducing, inhibiting or preventing microbial growth, comprising suitable and effective amounts of antimicrobial compositions in the desired end-user products.

Accordingly, it is an objective of the present invention to provide an antimicrobial composition comprising (i) raspberry ketone or its analogues; and (ii) at least one or more antimicrobial compounds not being raspberry ketone or its analogues. According to another objective of the present application, there is provided a synergistic antimicrobial composition of raspberry ketone or its analogues to kill or inhibit the growth of microorganisms in various aqueous and non-aqueous based end user compositions.

### SUMMARY OF THE INVENTION

The primary aspect of the present application is to provide an antimicrobial composition comprising: (a) about 0.01 to about 99.9 wt. % of at least one compound having a structure of formula (I) or formula (II):

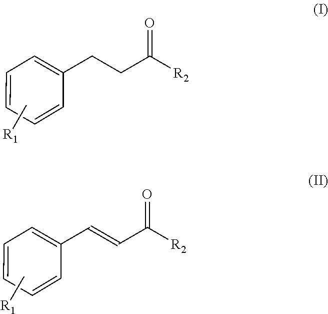

wherein R1 hydrogen, alkyl, alkoxy or hydroxyl; R2 is hydrogen, alkyl or alkoxy; and (b) about 0.1 to about 95% wt. % of one or more antimicrobial compounds not being compounds of formula (I) and formula (II).

Another non-limiting aspect of the present application provides an antimicrobial composition comprising: (a) about 0.01 to about 99.9 wt. % of at least one compound having a structure of formula (I) or formula (II) selected from the group consisting of:

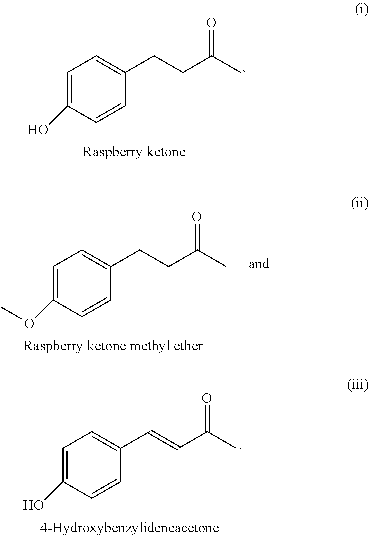

and (b) about 0.1 to about 95% wt. % of one or more antimicrobial compounds not being compounds of formula (I) and formula (II).

Yet another aspect of the present application discloses that the antimicrobial composition can be an aqueous composition or a non-aqueous composition.

Another aspect of the present application provides a process for preparing the above-described antimicrobial composition, wherein the process comprises the steps of mixing: (a) about 0.01 to about 99.9 wt. % of at least one compound having a structure of formula (I) or formula (II):

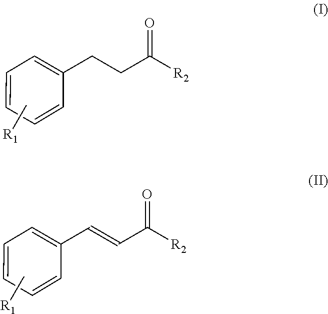

wherein R1 is hydrogen, alkyl, alkoxy or hydroxyl; R2 is hydrogen, alkyl or alkoxy; and (b) about 0.1 to about 95 wt. % of one or more antimicrobial compounds not being compounds of formula (I) and formula (II).

In another aspect, a method of killing or inhibiting the growth of bacteria and fungi in an aqueous or non-aqueous based end-user product selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms comprises incorporating the antimicrobial composition of the present application in an amount ranging from about 0.01 wt. % to 5.0 wt. % into the above desired products.

In yet another aspect, the present application provides an antimicrobial composition comprising (a) about 0.01 to about 40 wt. % of Raspberry ketone, Raspberry ketone methyl ether or 4-Hydroxybenzylideneacetone; and (b) about 0.1 to about 95 wt. % of one or more antimicrobial compounds selected from the group consisting of pentane-1,2-diol, hexane-1,2-diol, octane-1,2-diol, hexadecan-1-ol, citric acid, stearic acid, benzoic acid, anisic acid, cinnamic acid, phytic acid, caprylhydroxamic acid, hinokitiol, ethylhexylglycerin, hexyl glycerin, glyceryl caprylate/caprate, veratraldehyde, maltol, ethyl maltol and combinations thereof.

### DETAILED DESCRIPTION OF THE INVENTION

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “each independently selected from the group consisting of” means when a group appears more than once in a structure, that group may be selected independently each time it appears.

In a non-limiting embodiment, the present application discloses an antimicrobial composition comprising: (a) about 0.01 to about 99.9 wt. % of at least one compound having a structure of formula (I) or formula (II):

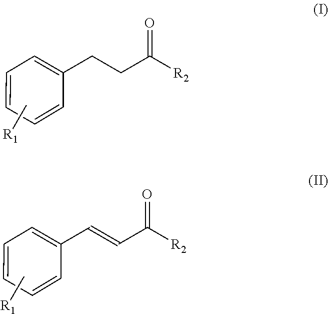

wherein R1 is hydrogen, alkyl, alkoxy or hydroxyl; R2 is hydrogen, alkyl or alkoxy; and (b) about 0.1 to about 95 wt. % of one or more antimicrobial compounds not being compounds of formula (I) and formula (II).

As used herein, the terms “antimicrobial”/“preservative” refer to substances capable of killing or inhibiting the growth of microorganisms, including but not limited, to bacteria and fungi.

As used herein, “Raspberry ketone”, also known as 4-(4-hydroxyphenyl) butan-2-one (HPB), having a CAS No: 5471-51-2, has been used as an aroma chemical in perfume industries, food industries and in compositions for weight loss with improved taste. Raspberry ketone was described for the first time as a characteristic component of Raspberry flavor (H. Schinz et. al. Helv. Chim. Acta. 1957, 40, 1839).

As used herein, “Raspberry ketone methyl ether”, also known as 4-(4-methoxyphenyl)butan-2-one (MPB) having a CAS No. 104-20-1, is a flavoring substance with a fruity, aromatic flavor note reminiscent of raspberry and blackberry as well as a fruity and raspberry taste used in food products.

As used herein, “4-Hydroxybenzylideneacetone”, also known as (E)-4-(4-hydroxyphenyl)but-3-en-2-one with a CAS No. 3160-35-8, is a precursor for raspberry ketone synthesis.

In some embodiments, compounds of formula (I) and formula (II) are selected from the group consisting of:

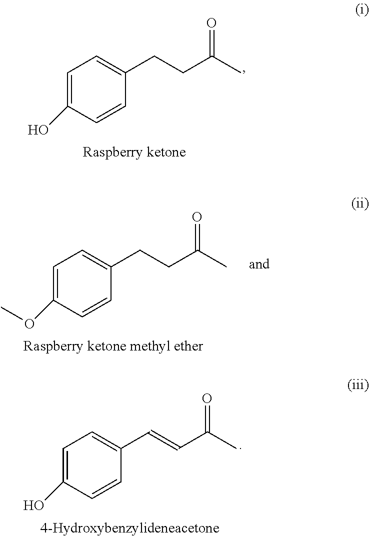

for the present application. The suitable amounts of compounds of formula (I) and formula (II) can be varied from about 0.01 wt. % to about 0.1 wt. %; or from 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; or from about 90 wt. % to about 95 wt. %; or from about 95 wt. % to about 99.9 wt. % based on the total weight of the antimicrobial composition.

As used herein, antimicrobial compound(s) not being compounds of formula (I) and formula (II) refers to any antimicrobial compound preferably selected from the group including, but not limited to, diols, organic acids or fatty acids, glycerins, caprylates, aldehydes, terpenes, terpenoids, essential oils, peptides, glucosides, enzymes, amino acids and their esters, sclerolide, sclareol and Camelia sinensis extracts and other conventional preservatives that are well-known to a person skilled in the pertinent art.

According to the present application, non-limiting examples of suitable antimicrobial diols useful herein include, but are not limited to, propanediol, butanediol, pentanediol, hexanediol, octanediol, nonanediol, decanediol and dodecanediol.

In another non-limiting embodiments, it is contemplated to use diols having a carbon chain length of from 3 to 12 atoms, including but not limited to, propane-1,2-diol, propane-1,3-diol, butane-1,2-diol, butane-1,3-diol, butane-1,4-diol, 2-methylpropane-1,2-diol, 2-methylpropane-1,3-diol, pentane-1,2-diol, pentane-1,3-diol, pentane-1,4-diol, pentane-1,5-diol, pentane-2,3-diol, pentane-2,4-diol, 2-methyl-pentane-2,4-diol, hexane-1,2-diol, hexane-1,3-diol, hexane-1,4-diol, hexane-1,5-diol, hexane-1,6-diol, hexane-2,3-diol, hexane-2,4-diol, hexane-2,5-diol, hexane-3,4-diol, heptane-1,2-diol, heptane-1,3-diol, heptane-1,4-diol, heptane-1,5-diol, heptane-1,6-diol, heptane-1,7-diol, heptane-2,3-diol, heptane-2,4-diol, heptane-2,5-diol, heptane-2,6-diol, heptane-3,4-diol, heptane-3,5-diol, octane-1,2-diol, octane-1,3-diol, octane-1,4-diol, octane-1,5-diol, octane-1,6-diol, octane-1,7-diol, octane-1,8-diol, octane-2,3-diol, octane-2,4-diol, octane-2,5-diol, octane-2,6-diol, octane-2,7-diol, octane-3,4-diol, octane-3,5-diol, octane-3,6-diol and octane-4,5-diol, nonane-1,9 diol, decane-1,2-diol, decane-1,10-diol, hexadecan-1-ol, and dodecane-1,12-diol.

In another non-limiting embodiment, the diols have a length of from 3 to 12 carbon atoms, including but not limited to, pentane-1,2-diol, hexane-1,2-diol, and octane-1,2-diol.

In another non-limiting embodiment, the organic acids or fatty acids or their salts or esters useful herein can be selected from acids having a carbon chain length of from 2 to 25 atoms. The carboxylic acids or fatty acids can be selected from the group including, but not limited to, propionic acid, acetic acid, benzoic acid, malonic acid, succinic acid, fumaric acid, maleic acid, adipic acid, lactic acid, stearic acid, levulinic acid, anisic acid, cinnamic acid, sorbic acid or tartaric acid, malic acid, gluconic acid, citric acid, caproic acid, perillic acid, phytic acid, salicylic acid, undecylenic acid, and the other acids include ascorbic acid, caprylhydroxamic acid, and sorbohydroxamic acid. These acids recited further enhance the antibacterial activity while not negatively affecting the quality of the end-user products in terms of their taste, texture, color and odor in which they are applied or engaged.

In another non-limiting embodiment, the organic acids or fatty acids or their salts or esters can be selected from carboxylic acids having from 2 to 25 carbon atoms, including but not limited to, citric acid, stearic acid, benzoic acid, anisic acid, cinnamic acid, phytic acid, sorbic acid, levulinic acid and other acids include caprylhydroxamic acid.

In another non-limiting embodiment, the glycerins useful herein include, but are not limited to, ethylhexylglycerin, butylglycerin, pentylglycerin, hexylglycerin, heptylglycerin, octylglycerin and cyclohexylglycerin.

In still another non-limiting embodiment, the caprylates useful herein include, but are not limited to, glyceryl mono-di caprylate, propylene mono-di caprylate, glyceryl caprylate, sorbitan caprylate, glyceryl undecylenate and glyceryl caprylate/caprate, isosorbide caprylate/caprate and stearyl caprylate.

Yet another non-limiting embodiment discloses employ aldehydes, including but not limited to, cinnamaldehyde, salicylaldehyde, veratraldehyde, benzaldehyde, butyraldehyde, propionaldehyde, acetaldehyde, and pyruvaldehyde.

The terpenes and terpenoids useful herein for the purposes of the present application include, but are not limited to, citral, pinene, nerol, b-ionone, geraniol, carvacrol, eugenol, carvone, terpeniol, anethole, camphor, menthol, limonene, nerolidol, farnesol, phytol, carotene, squalene, thymol, tocotrienol, perillyl alcohol, bomeol, myrcene, simene, carene, terpenene, tropolone, hinokitiol and linalool.

The essential oils useful herein include, but are not limited to, anise oil, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydrastis canadensis oil, berberidaceae daceae oil, ratanhia oil, Curcuma longa oil, sesame oil, macadamia nut oil, evening primrose oil, spanish sage oil, spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, bitter almond oil, palmarosa oil and vetiver oil.

As used herein, the conventional preservative compounds for the purposes of the present application are selected from the group including, but not limited to, benzoic acid and its sodium salt such as benzoic acid, sodium benzoate; salts of benzoic acid such as ammonium benzoate, butyl benzoate, calcium benzoate, ethyl benzoate, isobutyl benzoate, isopropyl benzoate, magnesium benzoate, MEA-benzoate, methyl benzoate, phenyl benzoate, potassium benzoate, propyl benzoate; propanoic acid and its salts such as propionic acid, ammonium propionate, calcium propionate, magnesium propionate, potassium propionate, sodium propionate; salicylic acid and its salts such as salicylic acid, calcium salicylate, magnesium salicylate, MEA-salicylate, sodium salicylate, potassium salicylate, TEA-salicylate; hexa-2,4-dienoic acid and its salts such as sorbic acid, calcium sorbate, sodium sorbate, potassium sorbate; biphenyl-2-ol as o-phenylphenol; inorganic sulphites and hydrogen sulphites such as sodium sulfite, ammonium bisulfite, ammonium sulfite, potassium sulfite, potassium hydrogen sulfite, sodium bisulfite, sodium meta bisulfite, potassium meta bisulfite; chlorobutanol; 4-hydroxybenzoic acid and its salts and esters other than the esters of isopropyl, isobutyl, phenyl, benzyl and pentyl such as 4-hydroxybenzoic acid, methylparaben, potassium ethylparaben, potassium paraben, sodium methylparaben, sodium ethylparaben, ethylparaben, sodium paraben, potassium methylparaben, calcium paraben; butyl 4-hydroxybenzoate and its salts and propyl 4-hydroxybenzoate and its salts such as butylparaben, propylparaben, sodium propoylparaben, sodium butylparaben, potassium butylparaben, potassium propylparaben; 3-acetyl-6-methylpyran-2,4(3H)-dione and its salts such as dehydroacetic acid, sodium dehydroacetate; formic acid, sodium formate; 3,3′-dibromo-4,4′-hexamethylene dioxydibenzamidine and its salts (including isethionate) such as dibromohexamidine isethionate; thimerosal; phenylmercuric salts such as phenyl mercuric acetate, phenyl mercuric benzoate; undec-10-enoic acid and its salts such as undecylenic acid, potassium undecylenate, sodium undecylenate, calcium undecylenate, MEA-undecylenate, TEA-undecylenate; 5-pyrimidinamine 1,3-bis(2-ethylhexyl)hexahydro-5-methyl-such as hexetidine; 5-bromo-5-nitro-1,3-dioxane; bronopol such as 2-bromo-2-nitropropane-1,3-diol; 2,4-dichlorobenzyl alcohol; 1-(4-chlorophenyl)-3-(3,4-dichlorophenyl)urea such as triclocarban; chlorocresol such as p-chloro-m-cresol, 5-chloro-2-(2,4-dichlorophenoxy)phenol such as triclosan; chloroxylenol; N,N″-methylenebis[N′-[3-(hydroxymethyl)-2,5-dioxoimidazolidin-4-yl]urea] such as imidazolidinyl urea; polyhexamethylene biguanide hydrochloride such as polyaminopropyl biguanide; 2-phenoxyethanol; methenamine; 1-(4-chlorophenoxy)-1-(imidazol-1-yl)-3,3-dimethylbutan-2-one such as climbazole; 1,3-bis(hydroxymethyl)-5,5-dim ethylimidazolidine-2,4-dione such as DMDM hydantoin; benzyl alcohol; 1-hydroxy methyl-6-(2,4,4-tri methylpentyl)-2 pyridon and its monoethanolamine salt such as 1-hydroxy-4-methyl-6-(2,4,4-trimethylpentyl)-2pyridon, piroctone olamine; 2,2′-methylenebis(6-bromo-4-chlorophenol) such as bromochlorophene; 4-isopropyl-m-cresol as o-cymen-5-ol; mixture of 5-chloro-2-methyl-isothiazol-3 (2H)-one and 2-methylisothiazol-3 (2H)-one with magnesium chloride and magnesium nitrate as methylchloroisothiazolinone and methylisothiazolinone; 2-chloroacetamide; N,N′-bis(4-chlorophenyl)-3,12-diimino-2,4,11,13-tetraazatetradeca nediamidine and its digluconate, diacetate and dihydrochloride such as chlorhexidine, chlorhexidine diacetate, chlorhexidine digluconate, chlorhexidine dihydrochloride; 1-phenoxypropan-2-ol such as phenoxyisopropanol; alkyl (C12-C22) trimethyl ammonium bromide and chloride such as behentrimonium chloride, cetrimonium bromide, cetrimonium chloride, laurtrimonium bromide, laurtrimonium chloride, steartrimonium bromide, steartrimonium chloride; 4,4-dimethyl-1,3-oxazolidine; N-(hydroxymethyl)-N-(dihydroxymethyl-1,3-dioxo-2,5-imidazolidinyl-4)-N′-(hydroxymethyl)urea such as diazolidinyl urea; benzenecarboximidamide 4,4′-(1,6-hexanediylbis(oxy))bis- and its salts (including isothionate and p-hydroxybenzoate) such as hexamidine, hexamidine diisethionate, hexamidine diparaben, hexamidine paraben; glutaraldehyde (pentane-1,5-dial) such as glutaral; 5-ethyl-3,7-dioxa-1-azabicyclo[3.3.0] octane such as 7-ethylbicyclooxazolidine; 3-(p-chlorophenoxy)-propane-1,2-diol such as chlorphenesin; sodium hydroxymethylamino acetate such as sodium hydroxymethylglycinate; silver chloride deposited on titanium dioxide; benzethonium chloride such as benzenemethanaminium,N,N-dimethyl-N-[2-[2-[4-(1,1,3,3,-tetramethylbutyl)phenoxy] ethoxy]ethyl]-chloride; benzalkonium chloride, benzalkonium bromide, benzalkonium saccharinate; methanol, (phenylmethoxy) such as benzylhemiformal; 3-iodo-2-propynylbutylcarbamate; 2-methyl-2H-isothiazol-3-one; ethyl lauroyl arginate HCl; citric acid (and) silver citrate such as 1,2,3-propanetricarboxylic acid, 2-hydroxy-, monohydrate and 1,2,3-propanetricarboxylic acid, 2-hydroxy-silver(1+) salt, monohydrate; and 4-(3-ethoxy-4-hydroxyphenyl)bu-tan-2-one.

According to the present application, other suitable antimicrobial agents useful herein include peptides, glucosides, enzymes, amino acids and their esters, sclerolide, sclareol, Camellia sinensis and plant extracts that are capable of killing or inhibiting microorganism and others known to a person skilled in the pertinent art.

Furthermore, according to the present application, other non-limiting antimicrobial agents useful herein include one or more antimicrobial compounds selected from the group consisting of propane-1,3-diol, pentane-1,2-diol, hexane-1,2-diol, octane-1,2-diol, hexadecan-1-ol, citric acid, stearic acid, benzoic acid, anisic acid, cinnamic acid, phytic acid, caprylhydroxamic acid, hinokitiol, ethylhexylglycerin, hexyl glycerin, glyceryl caprylate/caprate, veratraldehyde, maltol, ethyl maltol, phenyl propanol, tetradecyl trimethyl ammonium bromide (TTAB), 3-iodo-2-propynylbutyl-carbamate (IPBC), sodium fluoride and combinations thereof.

In some embodiments, the suitable range of one or more antimicrobial compounds not being compounds of formula (I) and formula (II) of the present application can be varied from about 0.01 wt. % to about 0.1 wt. %; or from 0.1 wt. % to about 1 wt. %; or from about 1 wt. % to about 2.5 wt. %; or from about 2.5 wt. % to about 5 wt. %; or from about 5 wt. % to about 10 wt. %; or 10 wt. % to about 15 wt. %; or from about 15 wt. % to about 20 wt. %; or from about 20 wt. % to about 25 wt. %; or from about 25 wt. % to about 30 wt. %; or from about 30 wt. % to about 35 wt. %; or from about 35 wt. % to about 40 wt. %; or from about 40 wt. % to about 45 wt. %; or from about 45 wt. % to about 50 wt. %; or from about 50 wt. % to about 55 wt. %; or from about 55 wt. % to about 60 wt. %; or from about 60 wt. % to about 65 wt. %; or from about 65 wt. % to about 70 wt. %; or from about 70 wt. % to about 75 wt. %; or from about 75 wt. % to about 80 wt. %; or from about 80 wt. % to about 85 wt. %; or from about 85 wt. % to about 90 wt. %; or from about 90 wt. % to about 95 wt. % based on the total weight of the antimicrobial composition.

The antimicrobial composition of the present application is useful for inhibiting or killing Staphylococcus aureus, Staphylococcus epidermidis, Streptococcus pneumoniae, Streptococcus pyogenes, Enterococcus faecalis, Haemophilus influenzae, Moraxella species, salmonella species, Campylobacter species, Pseudomonas aeruginosa, Clostridium botulinum, Clostridium perfringens, Corynebacteria species, Diplococci species, Mycobacteria species, Streptomyces species, Escherichia coli, Salmonella typhimurium, Salmonella enteritidis, Vibrio parahaemolyticus, Bacillus anthracis, Bacillus azotoformans, Bacillus cereus, Bacillus coagulans, Bacillus israelensis, Bacillus larvae, Bacillus mycoides, Bacillus polymyxa, Bacillus pumilis, Bacillus stearothormophillus, Bacillus subtilis, Bacillus thuringiensis, Bacillus validus, Bacillus weihenstephanensis, Bacillus pseudomycoides, Burkholderia cepacia, Burkholderia multivorans, Burkholderia cenocepacia, Burkholderia vietnamiensis, Burkholderia stabilis, Burkholderia ambifaria, Burkholderia dolosa, Burkholderia anthina, Burkholderia pyrrocinia, Candida tropicalis, Candida albicans, Hansenula anomala, Saccharomyces cerevisiae, Torulaspora delbreuckii, Zygosaccharomyces bailii, Zygosaccharomyces rouxii, Aspergillus niger, Aspergillus flavus, Aspergillus brasiliensis, Penicillium islandicum, Penicillium citrinum, Penicillium chrysogenum, Fusarium oxysporum, Fusarium graminearum, Fusarium solani, Alternaria alternata, and/or Mucor racemosus.

According to a non-limiting embodiment of the present application, the antimicrobial composition is used for killing or inhibiting the growth of Staphylococcus aureus, Escherichia coli, Burkholderia cepacia, Candida albicans, Pseudomonas aeruginosa, and Aspergillus brasihensis.

A different embodiment of the present application contemplates that the antimicrobial composition of the present application can be formulated as an emulsion, microemulsion, nanoemulsion, solution, dispersion, suspension, complex coacervate, or concentrate. The antimicrobial compositions can also include various optional additives. Examples of specific additives include, but are not limited to, colorants, pigments, plasticizers, surfactants, wetting agents, fillers, coloring agents, dispersing agents, thickening agents, rheology modifying agents, thixotropic agents, anti-freezing agents, co-solvents, pH modifying agents, ultraviolet light stabilizers, antioxidants, algaecides, antimicrobial agents, fragrances, buffers, hydrotropes, anti-soil agents, enzymes, suspending agents, emulsifying agent, anti-foaming agents, organic solvents, VOC-free solvents, solubilizers, and/or water-miscible solvents.

According to another non-limiting embodiment of the present application, the antimicrobial composition can provide a synergistic effect in various aqueous and non-aqueous based end-user applications, and wherein the synergistic index (SI) value is greater than 0.1 to less than 1.

Another non-limiting embodiment of the present application discloses that the antimicrobial composition can advantageously be used in personal care compositions, and wherein, the compositions can be an aqueous or non-aqueous based end-user composition. Aqueous and non-aqueous based end-user applications include, but are not limited to, personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products.

According to another non-limiting embodiment of the present application, the personal care compositions include, but are not limited to, sun care compositions, after-sun compositions, hair care compositions, conditioning compositions, skin care compositions, oral care compositions, face care compositions, lip care compositions, body care compositions, nail care compositions, anti-aging compositions, deodorant compositions, color cosmetic compositions, color-protection compositions, self-tanning compositions, and foot care compositions.

In non-limiting embodiments, the present application discloses that suitable ranges of incorporating the above-described antimicrobial composition for killing or inhibiting the growth of bacteria and fungi in aqueous or non-aqueous based end-user products can be varied from about 0.01 wt. % to about 0.1 wt. %, or from about 0.1 wt. % to about 1 wt. %, or from about 1 wt. % to about 2.5 wt. %, or from about 2.5 wt. % to about 5 wt. % based on the total weight of the aqueous or non-aqueous personal care composition.

In a non-limiting embodiment, the present application discloses an antimicrobial composition comprising (a) about 0.01 to about 40 wt. % of Raspberry ketone, Raspberry ketone methyl ether or 4-Hydroxybenzylideneacetone; and (b) about 0.1 to about 95 wt. % of one or more antimicrobial compounds selected from the group consisting of propane-1,3-diol, pentane-1,2-diol, hexane-1,2-diol, octane-1,2-diol, hexadecan-1-ol, citric acid, stearic acid, benzoic acid, anisic acid, cinnamic acid, phytic acid, caprylhydroxamic acid, hinokitiol, ethylhexylglycerin, hexyl glycerin, glyceryl caprylate/caprate, veratraldehyde, maltol, ethyl maltol, phenyl propanol, tetradecyl trimethyl ammonium bromide (TTAB), 3-iodo-2-propynylbutyl-carbamate (IPBC), sodium fluoride and combinations thereof.

Another embodiment of the present application provides a process for preparing antimicrobial compositions, wherein the process comprises the steps of mixing: (a) about 0.01 to about 99.9 wt. % of at least one compound having a structure of formula (I) or formula (II):

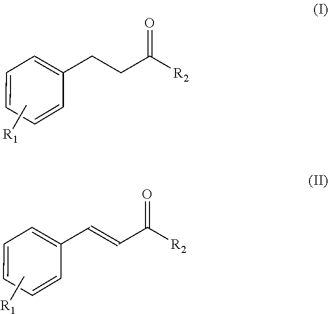

wherein R1 is hydrogen, alkyl, alkoxy or hydroxyl; R2 is hydrogen, alkyl or alkoxy; and (b) about 0.01 to about 95 wt. % of one or more antimicrobial compounds not being compounds of formula (I) and formula (II).

Yet another embodiment of the present application discloses a method of killing or inhibiting the growth of bacteria and fungi in aqueous or non-aqueous based end-user products selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms comprising incorporating about 0.01 wt. % to 5.0 wt. % of the above-described antimicrobial composition into the desired products.

Further, certain aspects of the present application are illustrated in detail by way of the following examples. The examples are given herein for illustration of the application and are not intended to be limiting thereof.

### EXAMPLES

### Example 1: Minimal Inhibitory Concentrations (MICs)

Example 1 (Table 1) demonstrates activity of each active versus six common microbial contaminants: Staphylococcus aureus 6538, Escherichia coli 8739, Burkholderia cepacia 25416 Pseudomonas aeruginosa 9027, Candida albicans 10231, and Aspergillus brasiliensis 16404). Briefly, each active was dissolved and then serially diluted in DMSO using a 96-well dilution microplate. Automated liquid handler was then used to stamp multiple assay plates out of this dilution plate. The appropriate growth medium containing a desired microorganism was then added to each well of the plate. For bacterial strains, Trypticase Soy Broth (TSB) was used with the final cell density of the microorganisms, in each well, being 10⁶ cfu/ml. For fungal strains, Yeast Malt Broth (YMB) was used with the final density of each microorganism being 10⁵ cfu/ml. Bacterial plates were incubated for 48 hours at 35° C. and fungal plates were incubated for 4-5 days at 28° C. prior to being evaluated. The lowest concentration of each compound to inhibit any visible growth was defined as minimum inhibitory concentration (MIC). In some cases, the highest tested concentration of the active was limited by its solubility in water. Whenever MIC was greater than the highest tested concentration, it was reported as being equal to, or greater than (>) the previous hypothetical dilution.

TABLE 1
Minimal Inhibitory Concentrations (MICs) mean values in ppm

| | | | | P. | C. | A. |
|---|---|---|---|---|---|---|
| Antimicrobial | S. | E. | B. | aeru- | al- | brasili- |
| compound | aureus | coli | cepacia | ginosa | bicans | ensis |
| Raspberry ketone | 28,000 | 4,000 | 2,000 | 4,000 | 4,000 | 4,000 |
| Raspberry ketone | 28,000 | 2,000 | 1,000 | 4,000 | 2,000 | 1,000 |
| methyl ether | | | | | | |
| 4-Hydroxybenzyl- | 500 | 2,000 | 1,000 | 4,000 | 500 | 1,000 |
| ideneacetone | | | | | | |

### Example 2: Synergy Between 4-(4-hydroxyphenyl)butan-2-one (HPB) and Other Antimicrobials

Synergy between of 4-(4-hydroxyphenyl)butan-2-one (HPB) and various known antimicrobial compounds was demonstrated versus Staphylococcus aureus 6538, Burkholderia cepacia 25416, and Aspergillus brasiliensis 16404 (Table 2a-2c). The synergistic antimicrobial effect was determined using a commonly accepted method described by Kull A. C, Eisman, P. C. Sylwestrowicz, H. D. and Mayer, R. L. 1961. Applied Microbiology, 9:538-541

Briefly, the standard checkerboard approach was used to make all the dilutions. HPB (compound A) solution was prepared and then serially diluted in DMSO using the 96-well dilution plate. Automated liquid handler was then used to stamp multiple assay plates using this dilution plate. Each well of the assay plate was then supplemented with a predetermined concentration of the second antimicrobial (compound B). The final concentration of DMSO was kept constant across the plate for each assay, not exceeding 3% (w/w).

To evaluate antifungal activity of the actives, freshly prepared A. brasiliensis spores were harvested and resuspended in Yeast and Malt Broth (YMB). The spore suspension was then added to the appropriate plates with the final density being 10⁵ cfu/ml.

To evaluate antibacterial activity of the actives, freshly prepared S. aureus and B. cepacia cells were harvested and resuspended in Trypticase Soy Broth (TSB). The cell suspension was then added to the wells of the appropriate plates with the final cell density being 10⁶ cfu/ml. Fungal plates were incubated at 28° C. for 4-5 days, prior to being evaluated. Bacterial plates were incubated for 48 hours at 35° C. prior to being evaluated.

The Minimal Inhibitory Concentration (MIC) of the combination A+B (in different ratios) was then compared to the MICs of the compound A and the B acting alone by calculating the Synergy Index (SI) using formula below.

Synergy Index (SI)=Qa/QA+Qb/QB
Qa is the MIC of compound A (HPB) in ppm, when compound A is being used in combination with compound B
QA is the MIC of compound A (HPB) in ppm, acting alone
Qb is the MIC of compound B in ppm, when the compound B is being used in combination with compound A
QB is the MIC of compound B in ppm, acting alone
   Each ratio Qa/QA and Qb/QB is sometimes referred to as Fractional Inhibitory Concentrations (FICs) of compounds A and B, respectively, when these two compounds are being used in combination with one another. The sum of FICs constitute the Synergy Index (SI).
   The synergistic effect is indicated if the Synergy Index is below one (SI<1). An additive effect is indicated if the Synergy Index is equal to one (SI=1). An antagonistic effect is indicated if synergy index is greater than one (SI>1). The lower the SI index the greater the synergy.

TABLE 2a
Synergy data vs. A. brasiliensis, generated using the checkerboard microplate
assay with the compound A being 4-(4-hydroxyphenyl)butan-2-one (HPB)

| | | MIC in ppm | | | | | |
|---|---|---|---|---|---|---|---|
| A. | | | Com- | | Com- | | |
| brasili- | | HPB | pound | HPB | pound | SI | Inter- |
| ensis | Compound B | (Qa) | B (Qb) | (QA) | B (Qb) | index | action |
| | Phenoxyethanol | 2,000 | 800 | 4,000 | 3,125 | 0.75 | Synergistic |
| | Phytic acid | 2,000 | 3,125 | 4,000 | 12,500 | 0.75 | Synergistic |
| | EDTA | 2,000 | 800 | 4,000 | 12,500 | 0.56 | Synergistic |
| | GLDA | 2,000 | 1,600 | 4,000 | 6,250 | 0.75 | Synergistic |
| | Glyceryl | 2,000 | 800 | 4,000 | 3,125 | 0.75 | Synergistic |
| | caprylate/caprate | | | | | | |
| | Ethylhexylglycerin | 2,000 | 100 | 4,000 | 1,600 | 0.56 | Synergistic |
| | Hexyl glycerin | 2,000 | 1,600 | 4,000 | 3,125 | 1 | Additive |
| | 1,3-Propanediol | 2,000 | 100,000 | 4,000 | ≥200,000 | ≤1 | Additive |
| | 1,2-Pentanediol | 2,000 | 50,000 | 4,000 | 100,000 | 1 | Additive |
| | 1,2-Hexanediol | 2,000 | 6,300 | 4,000 | 12,500 | 1 | Additive |
| | 1,2-Octanediol | 2,000 | 100 | 4,000 | 1,600 | 0.56 | Synergistic |
| | Cinnamic acid | 4,000 | 500 | 4,000 | 500 | 1-2 | Additive |
| | Benzoic acid | 1,000 | 500 | 4,000 | 1,000 | 0.75 | Synergistic |
| | Anisic acid | 2,000 | 250 | 4,000 | 1,000 | 0.75 | Synergistic |
| | Benzyl alcohol | 1,000 | 2,500 | 4,000 | 5,000 | 0.75 | Synergistic |
| | Tetradecyl trimethyl | 1,000 | 3.1 | 4,000 | 25 | 0.375 | Synergistic |
| | ammonium bromide | | | | | | |
| | (TTAB) | | | | | | |
| | Caprylhydroxamic | 250 | 125 | 4,000 | 250 | 0.5625 | Synergistic |
| | acid | | | | | | |
| | Veratraldehyde | 500 | 1,000 | 4,000 | 2,000 | 0.625 | Synergistic |
| | Maltol | 500 | 1,000 | 4,000 | 2,000 | 0.75 | Synergistic |

TABLE 2b
Synergy data vs. A aureus, generated using the checkerboard microplate assay
with the compound A being 4-(4-hydroxyphenyl)butan-2-one (HPB)

| | | MIC in ppm | | | | | |
|---|---|---|---|---|---|---|---|
| | | | Com- | | Com- | | |
| S. | | HPB | pound | HPB | pound | SI | Inter- |
| aureus | Compound B | (Qa) | B (Qb) | (QA) | B (QB) | index | action |
| | Benzyl alcohol | 4,000 | 625 | ≥8,000 | 5,000 | ≤0.625 | synergistic |
| | Phenyl propanol | 4,000 | 156 | ≥8,000 | 2,500 | ≤0.5625 | synergistic |
| | Tetradecyl trimethyl | 2,000 | 0.4 | ≥8,000 | 1.6 | ≤0.5 | synergistic |
| | ammonium bromide | | | | | | |
| | (TTAB) | | | | | | |
| | Phenethyl alcohol | 2,000 | 2,000 | ≥8,000 | ≥4,000 | ≤0.75 | synergistic |
| | Caprylhydroxamic acid | 4,000 | 125 | ≥8,000 | 1,000 | ≤0.625 | synergistic |
| | Veratraldehyde | 4,000 | 1,000 | ≥8,000 | ≥8,000 | ≤0.625 | synergistic |

TABLE 2c
Synergy data vs. B. cepacia, generated using the checkerboard microplate assay
with the compound A being 4-(4-hydroxyphenyl)butan-2-one (HPB)

| | | MIC in ppm | | | | | |
|---|---|---|---|---|---|---|---|
| | | | Com- | | Com- | | |
| B. | | HPB | pound | HPB | pound | SI | Inter- |
| cepacia | Compound B | (Qa) | B (Qb) | (QA) | B (Qb) | index | action |
| | Phenyl propanol | 1,000 | 156 | 2,000 | 625 | 0.75 | synergistic |
| | Sodium fluoride | 250 | 10,000 | 2,000 | ≥200,000 | ≤0.625 | synergistic |
| | Tetradecyl trimethyl | 1,000 | 6.3 | 2,000 | 50 | 0.625 | synergistic |
| | ammonium bromide | | | | | | |
| | (TTAB) | | | | | | |
| | lodopropynyl | 1,000 | 2 | 2,000 | 250 | 0.508 | synergistic |
| | butylcarbamate (IPBC) | | | | | | |
| | Phenethyl alcohol | 1,000 | 125 | 2,000 | 1,000 | 0.625 | synergistic |
| | Caprylhydroxamic acid | 1,000 | 31.3 | 2,000 | 125 | 0.75 | synergistic |
| | Veratraldehyde | 500 | 1000 | 2,000 | 2,000 | 0.75 | synergistic |

### Example 3: Synergy Between 4-(4-hydroxyphenyl)butan-2-one (HPB) and Other Antimicrobials Using the Time Kill Assay

This example demonstrates that the given combination of the antimicrobials results in the faster kill rate of microorganisms compared to the kill rates of each antimicrobial being used alone.

Data demonstrating synergy between MPB and other actives in the formulated product, was generated using the non-ionic emulsion base (Table 3) following a 21-day double inoculation challenge test. Briefly, samples containing a single active or a combination of two actives were prepared by post-adding the actives to the emulsion base.

The composite inoculum consisted of four bacterial species (Staphylococcus aureus 6538, Escherichia coli 8739, Burkholderia cepacia 25416, and Pseudomonas aeruginosa 9027), and two fungal species (Candida albicans 10231 and Aspergillus brasiliensis 16404); six microbial species altogether. The test samples were spiked with the microbial inoculum on days 0 and 14, with the final concentration of bacteria being 10⁶ cfu/g and the final concentration of fungi being 10⁵ cfu/g. Inoculated samples were incubated at 28° C. On days 2, 7, 14 and 21 the samples were neutralized and plated to recover viable microorganisms. Letheen Agar was used for the recovery of bacteria and Potato Dextrose Agar with 0.1% chloramphenicol was used for the recovery of fungi.

TABLE 3
Composition of the non-ionic emulsion base used in Example 3

| | |
|---|---|
| Ingredients | % w/w |

PHASE A

| | |
|---|---|
| Stearic acid, NF | 5.0 |
| Mineral Oil | 2.5 |
| Cetyl Alcohol (hexadecan-1-ol) | 1.0 |
| Ceteareth-5 | 0.5 |
| PEG 100 Stearate | 1.5 |

PHASE B

| | |
|---|---|
| DI water | 86.9 |
| Triethanolamine 99% | 1.0 |

PHASE C

| | |
|---|---|
| Citric Acid 30% aq. | 0.6 |

TABLE 4
Synergy data generated in the non-ionic pH 7 emulsion
base after 48 hours of incubation

| | | Cell counts | Log reduction |
|---|---|---|---|
| | | recovered after | compared to the |
| | | 48 hours log | unpreserved |
| Sample description | Microorganisms | (cfu/ml) | control |
| Unpreserved sample | Bacteria | 6.0 | N/A |
| | Fungi | 4.4 | N/A |
| 0.25% HPB | Bacteria | 5.4 | 0.6 |
| | Fungi | 4.5 | 0.0 |
| 0.5% Phenoxyethanol | Bacteria | 4.5 | 1.5 |
| (PE) | Fungi | 4.2 | 0.2 |
| 0.5% PE + 0.25% | Bacteria | <2.0 | >4.0 |
| HPB | Fungi | 4.0 | 0.4 |

Results shown in Table 4 demonstrate that 0.25% HPB produces only a 0.6-log reduction in total bacterial cell count after 48 hours of incubation. Similarly, 0.5% phenoxyethanol produces a 1.5-log reduction in bacterial cell counts within the same time period. One would expect to see the 2.1-log reduction if these two actives in the specified concentrations were combined. However, when 0.25% HPB was combined with 0.5% phenoxyethanol, unexpectedly, at least a 4-log reduction in bacterial cell count is seen after the 48-hour time period. This signifies synergy between HPB and phenoxyethanol.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

What is claimed is:

1. An antimicrobial composition comprising:
(a) about 0.01 to about 99.9 wt. % of at least one compound having a structure of formula (I) or formula (II): 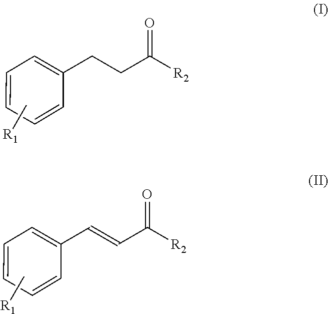
wherein R₁ is hydrogen, alkyl, alkoxy or hydroxyl; R₂ is hydrogen, alkyl or alkoxy; and
(b) about 0.1 to about 95% wt. % of one or more antimicrobial compounds not being compounds of formula (I) and formula (II).

2. The antimicrobial composition according to claim 1, wherein the compound of formula (I) and formula (II) is selected from the group consisting of:

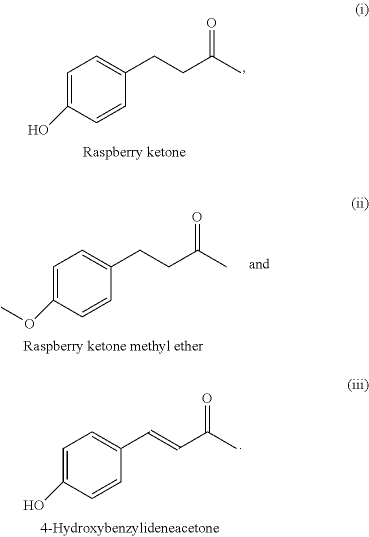

3. The antimicrobial composition according to claim 1, wherein the antimicrobial compound (b) is selected from the group consisting of diols, organic acids including carboxylic acids, glycerins, caprylates, aldehydes, terpenes, terpenoids, essential oils, peptides, glucosides, enzymes, amino acids and their esters, sclerolide, sclareol, and Camelia sinensis extracts.

4. The antimicrobial composition according to claim 3, wherein the diol is selected from the group consisting of propanediol, butanediol, pentanediol, hexanediol, octanediol, nonanediol, decanediol and dodecanediol.

5. The antimicrobial composition according to claim 3, wherein the organic acid is selected from the group consisting of benzoic acid, sorbic acid, levulinic acid, anisic acid, cinnamic acid, perillic acid, phytic acid, salicylic acid, propionic acid, lactic acid, undecylenic acid, caprylhydroxamic acid and sorbohydroxamic acid.

6. The antimicrobial composition according to claim 3, wherein the glycerin is selected from the group consisting of ethylhexylglycerin, hexyl glycerin and cyclohexyl glycerin.

7. The antimicrobial composition according to claim 3, wherein the caprylate is selected from the group consisting of glyceryl mono-di caprylate, propylene mono-di caprylate, glyceryl caprylate, sorbitan caprylate, glyceryl undecylenate and glyceryl caprylate/caprate.

8. The antimicrobial composition according to claim 3, wherein the aldehyde is selected from the group consisting of cinnamaldehyde, salicylaldehyde, veratraldehyde and benzaldehyde.

9. The antimicrobial composition according to claim 3, wherein the antimicrobial compound is selected from the group consisting of phenoxyethanol, benzyl alcohol, alkyl parabens, bronopol, formaldehyde, diazolidinyl urea, imidazolidinyl urea, sodium hydroxymethyl glycinate, TMAD, DMDMH, silver, chlorphenesin, nisin, natamycin, triclosan, 2-methyl-4-isothiazolin-3-one (MIT), 1,2-benzisothiazolin-3-one (BIT), 5-chloro-2-methyl-4-isothiazolin-3-one (CMIT), 2-octyl-4-isothiazolin-3-one (OIT), 3-iodo-2-propynylbutyl-carbamate (IPBC), 3-iodopropynyl-N-phenyl carbamate (IPPC), zinc pyrithione (ZnPy), quaternary ammonium compounds, hydantoins, sodium pyrithione, phenyl ethanol, phenyl propanol, alkyl (C12-C22) trimethyl ammonium bromide, sodium fluoride and benzalkonium quaternary ammonium.

10. The antimicrobial composition according to claim 9, wherein the antimicrobial compound is selected from the group consisting of phenyl propanol, alkyl (C12-C22) trimethyl ammonium bromide, tetradecyl trimethyl ammonium bromide (TTAB), 3-iodo-2-propynylbutyl-carbamate (IPBC) and sodium fluoride.

11. The antimicrobial composition according to claim 1, wherein the composition is used for killing or inhibiting the growth of Staphylococcus aureus, Staphylococcus epidermidis, Streptococcus pneumoniae, Streptococcus pyogenes, Enterococcus faecalis, Haemophilus influenzae, Moraxella species, salmonella species, Campylobacter species, Pseudomonas aeruginosa, Clostridium botulinum, Clostridium perfringens, Corynebacteria species, Diplococci species, Mycobacteria species, Streptomyces species, Escherichia coli, Salmonella typhimurium, Salmonella enteritidis, Vibrio parahaemolyticus, Bacillus anthracis, Bacillus azotoformans, Bacillus cereus, Bacillus coagulans, Bacillus israelensis, Bacillus larvae, Bacillus mycoides, Bacillus polymyxa, Bacillus pumilis, Bacillus stearothormophillus, Bacillus subtilis, Bacillus thuringiensis, Bacillus validus, Bacillus weihenstephanensis, Bacillus pseudomycoides, Burkholderia cepacia, Burkholderia multivorans, Burkholderia cenocepacia, Burkholderia vietnamiensis, Burkholderia stabilis, Burkholderia ambifaria, Burkholderia dolosa, Burkholderia anthina, Burkholderia pyrrocinia, Candida tropicalis, Candida albicans, Hansenula anomala, Saccharomyces cerevisiae, Torulaspora delbreuckii, Zygosaccharomyces bailii, Zygosaccharomyces rouxii, Aspergillus niger, Aspergillus flavus, Aspergillus brasiliensis, Penicillium islandicum, Penicillium citrinum, Penicillium chrysogenum, Fusarium oxysporum, Fusarium graminearum, Fusarium solani, Alternaria alternata and/or Mucor racemosus.

12. The antimicrobial composition according to claim 1, wherein the composition is used for killing or inhibiting the growth of Staphylococcus aureus, Escherichia coli, Burkholderia cepacia, Candida albicans, Pseudomonas aeruginosa and Aspergillus brasiliensis.

13. The antimicrobial composition according to claim 1, wherein the composition is aqueous or non-aqueous.

14. The antimicrobial composition according to claim 1, wherein the composition is formulated as an emulsion, microemulsion, nanoemulsion, solution, dispersion, suspension, complex coacervate or concentrate.

15. The antimicrobial composition according to claim 1, wherein the composition provides a synergistic effect having a synergistic index (SI) value greater than 0.1 to less than 1.

16. Use of the antimicrobial composition of claim 1, in an aqueous or non-aqueous based end-user application selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products.

17. The use of the antimicrobial composition according to claim 16, wherein the personal care or cosmetic composition is selected from sun care compositions, after-sun compositions, hair care compositions, conditioning compositions, skin care compositions, oral care compositions, face care compositions, lip care compositions, body care compositions, nail care compositions, anti-aging compositions, deodorant compositions, color cosmetic compositions, color-protection compositions, self-tanning compositions and foot care compositions.

18. The use of the antimicrobial composition according to claim 13, wherein the amount of said composition employed in an aqueous or non-aqueous based end-user composition is in the range of from about 0.01 wt. % to about 5.0 wt. % of the total composition.

19. A process for preparing the antimicrobial composition of claim 1, wherein the process comprises the steps of mixing:
(a) about 0.01 to about 99.9 wt. % of at least one compound having a structure of formula (I) or formula (II): 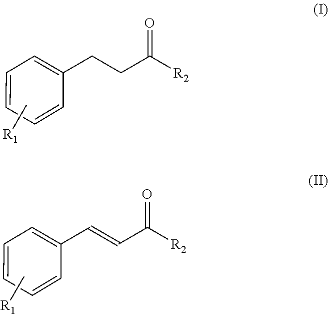
wherein R₁ is hydrogen, alkyl, alkoxy or hydroxyl; and R₂ is hydrogen, alkyl, or alkoxy; and
(b) about 0.1 to about 95 wt. % of one or more antimicrobial compounds not being compounds of formula (I) and formula (II).

20. A method of killing or inhibiting the growth of bacteria and fungi in an aqueous or non-aqueous based end-user product selected from the group consisting of personal care or cosmetic products, toiletry products, oral care products, skin care products, hair care products, household & cleaning products, soap and bath products, industrial and institutional cleaning products, disinfecting products, wound care products, sanitary products, agricultural compositions, textile products, coating products and laundry products that are susceptible to growth of microorganisms, comprising incorporating about 0.01 wt. % to 5.0 wt. % of the antimicrobial composition of claim 1 into said product.

21. An antimicrobial composition comprising:
(a) about 0.01 to about 40 wt. % of Raspberry ketone, Raspberry ketone methyl ether or 4-Hydroxybenzylideneacetone; and
(b) about 0.1 to about 95 wt. % of one or more antimicrobial compounds selected from the group consisting of propane-1,3-diol, pentane-1,2-diol, hexane-1,2-diol, octane-1,2-diol, hexadecan-1-ol, citric acid, stearic acid, benzoic acid, anisic acid, cinnamic acid, phytic acid, caprylhydroxamic acid, hinokitiol, ethylhexylglycerin, hexyl glycerin, glyceryl caprylate/caprate, veratraldehyde, maltol, ethyl maltol, phenyl propanol, tetradecyl trimethyl ammonium bromide (TTAB), 3-iodo-2-propynylbutyl-carbamate (IPBC), sodium fluoride and combinations thereof.
